(19) United States
(12) Patent Application Publication
(10) Pub. No.: US 2009/0191276 A1
(43) Pub. Date: Jul. 30, 2009

(54) COLLOIDOSOMES HAVING TUNABLE PROPERTIES AND METHODS FOR MAKING COLLOIDOSOMES HAVING TUNABLE PROPERTIES

(75) Inventors: Jin W. Kim, Yongin-si (KR); David A. Weltz, Bolton, MA (US)

Correspondence Address: Harvard University & Medical School;c/o Wolf, Greenfield & Sacks, P.C., 600 Atlantic Avenue, Boston, MA, 02210-2206, US

(73) Assignee: Fellows and President of Harvard University, Cambridge, MA (US)

(21) Appl. No.: 12/019,454

(22) Filed: Jan. 24, 2008

(51) Int. Cl. A61K 9/50; B01J 13/02; A61K 38/43; B32B 15/02

(52) U.S. Cl. 424/499; 264/4.1; 264/4.33; 264/4.3; 428/402.21; 428/402.2; 424/94.1

(57) ABSTRACT

Colloidosomes having tunable properties, methods for making the same, and applications thereof are described. Colloidosomes described herein are responsive to certain external stimulus to alter one or more properties of the colloidosome. Methods for making colloidosomes include forming a shell of colloidal particles on a core material where the colloidal particles and the core material have attractive interactions.

### STATEMENT REGARDING FEDERALLY SPONSORED RESEARCH OR DEVELOPMENT

The present invention was made with United States government support under Grant No. DMR-0602684 awarded by the National Science Foundation. The United States government may have certain rights in this invention.

### COPYRIGHT NOTICE

This patent disclosure may contain material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### INCORPORATION BY REFERENCE

All patents, patent applications and publications cited herein are hereby incorporated by reference in their entirety in order to more fully describe the state of the art as known to those skilled therein as of the date of the invention described herein.

### BACKGROUND OF THE INVENTION

Colloidosomes are hollow shells composed of closely-packed colloidal particles. Colloidosomes may be formed when colloidal particles arrange in the form of a shell at an interface between two surfaces, leading to interesting encapsulation properties. The permeability of encapsulated species in colloidosomes is dependent on the size of pores between the particles in the shell. To date, colloidosomes have been formed by emulsifying two immiscible fluids, such as oil and water, allowing colloidal particles to assemble at the interface between the two immiscible fluids. The colloidal particles assemble at the interface between the two immiscible fluids to minimize the overall interfacial free energy. However, the conventional method for forming colloidosomes is a delicate process that can be easily disrupted. For example, if the thermal fluctuations in the system are large enough to overcome the minimum in the overall interfacial energy, assembly of the colloidal particles at the interface can be disrupted.

Moreover, the colloidosomes that result generally do not have close-packing of the colloidal particles. Generally, the resulting colloidosomes have high permeability to low molecular weight macromolecules and nanoscale species due to the large interstitial voids typically obtained with colloidal particle packing in the liquid phase. Moreover, the release of larger encapsulated material from such traditional colloidosomes relies on external triggers such as changes in osmotic pressure or mechanical forces to crush or break open the capsule, which precludes precise control of the release response.

### SUMMARY OF THE INVENTION

Colloidosomes having tunable properties (i.e., properties that can be altered upon application of one or more external stimuli) and methods for making and/or using the colloidisomes having tunable properties are provided. The colloidosomes provide control of the permeability to both small and large species to be captured by or released from the colloidosome. In accordance with certain embodiments, colloidosomes described herein can be responsive to certain stimuli, such as temperature, electric field, swelling agents, and the like. In response to certain stimuli, the colloidosomes can alter their properties, such as permeability, mechanical properties, morphology, and the like. For example, the responsiveness of the colloidisomes to external stimuli help control the colloidosome shell quality, shell permeability, and release characteristics.

Methods for making colloidosomes having tunable properties are also described herein. In accordance with certain embodiments, methods for making colloidosomes having tunable properties include providing a core material and colloidal particles having attractive interactions between the core material and the colloidal particles. The attractive interactions can include electrostatic interactions, magnetic interactions, pyroelectric interactions, and the like. The core material can include a gel, such as a hydrogel, and the like, which provides a feature for assembly of colloidal particles on the core. The method further includes allowing the colloidal particles to assemble on the core.

In certain embodiments, colloidosomes having tunable properties can include a core polymer gel material and a collection of colloidal particles assembled on a surface of the core material, wherein the core polymer gel material surface and the colloidal particles possess at least one attractive interaction. In certain embodiments, the core polymer gel material can be responsive to an external stimulus that alters the interparticle distance between colloidal particles. the initial diameter of the core polymer gel material.

In certain embodiments, methods for changing at least one property of a colloidosome is described. The method can include applying one or more external stimuli to a colloidosome, where the colloidosome includes a core gel material and a shell of colloidal particles formed on the core gel material. Moreover, the core material and the colloidal particles can possess at least one attractive interaction, and the core material can respond to the one or more external stimuli to change the interparticle distance between the colloidal particles.

In certain embodiments, a drug delivery vehicle is described herein. The drug delivery vehicle can include a core polymer gel material and a collection of colloidal particles assembled on a surface of the core material. The drug delivery vehicle can further include an active agent encapsulated in the core polymer gel material. In certain embodiments, the core polymer gel material surface and the colloidal particles can possess at least one attractive interaction. Moreover, the core polymer gel material can be responsive to an external stimulus that alters the interparticle distance between colloidal particles.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a schematic diagram of a negatively-charged colloidal particle assembled onto a positively-charged core material in accordance with certain embodiments;

FIGS. 2A through 2C schematically show a colloidosome undergoing morphological changes to change the permeability of the colloidosome in response to a stimulus, e.g., heat, applied in accordance with certain embodiments;

FIG. 3A is an image showing a colloidosome having polystyrene colloidal particles assembled on a gel core material in accordance with certain embodiments;

FIG. 3B is an image showing a colloidosome at a temperature higher than that shown in FIG. 3A where the contraction of the gel leads to packing of the polystyrene colloidal particles in accordance with certain embodiments;

FIG. 3C is an image showing a buckled colloidosome at a temperature higher than that shown in FIG. 3B where the contraction of the gel leads to buckling or distortion of the polystyrene colloidal particles layer in accordance with certain embodiments;

FIG. 4A shows an electron microscope image of a buckled colloidosome after the colloidosome was dried and fractured in accordance with certain embodiments;

FIG. 4B is a graph of the buckled layer thickness as a function of the particle diameter over two orders of magnitude in accordance with certain embodiments;

FIG. 5A is a graph of the ratio of diameter to initial diameter of the gel core structure as a function of temperature showing the temperature at which contraction of the core gel material and buckling occurs for a gel colloidosome having 500 nm polystyrene particles, 80 nm particles, and 20 nm particles in accordance with certain embodiments;

FIG. 5B shows a graph of the diameter as a function of temperature showing the temperature at which contraction of the core material and buckling occurs for two colloidosomes having 80 nm polystyrene particles but with different diameter gel as the core material in accordance with certain embodiments;

FIG. 6 is a time series of fluorescence images illustrating the permeation of fluorescein sodium salts into a plain gel (top row) and colloidosomes made with 1 μm PS particles (middle row) and 80 nm PS particles (bottom row) in accordance with certain embodiments; and

FIG. 7 is a graph of the intensity of fluorescein detected as a function of time for a gel colloidosome made with 1 μm PS particles, 200 nm PS particles, 80 nm PS particles, and 310 nm PBMA particles in accordance with certain embodiments.

### DETAILED DESCRIPTION OF THE INVENTION

Methods for making colloidosomes having tunable properties are described herein. In accordance with certain embodiments, methods for making colloidosomes having tunable properties include providing a core material and colloidal particles having attractive interactions with the core material. The attractive interactions can include electrostatic interactions, magnetic interactions, pyroelectric interactions, and the like. The method further includes allowing the colloidal particles to assemble on the core. In certain embodiments, the attractive interactions can be stronger than thermal fluctuations or other factors that tend to disrupt close and/or ordered packing of particles on a surface, so that the colloidal particles assembly onto the core material to obtain a uniform shell of colloidal particles around the core material.

Without wishing to be bound by theory, the attractive interactions may overcome some of the difficulties associated with assembly of conventional immiscible liquid-liquid (oil-water) interfaces. Generally, immiscible fluids form one or more interfaces in order to minimize the overall interfacial free energy. When colloidal particles are introduced into the system, the colloidal particles can migrate and assemble at the interface to further minimize the overall interfacial free energy. However, the system is at a metastable equilibrium and any external forces that are large enough to perturb the system out of the interfacial free energy minimum can disrupt the assembly of the colloidal particles at the interface. In certain conventional systems, even thermal fluctuations may be sufficient to overcome the free energy minimum and disrupt the formation of stable colloidosome structures.

In contrast, the attractive interactions between the core material and the colloidal particles described herein can be stronger than thermal fluctuations that may be present in the system, so that the colloidal particles assemble onto the core material as a uniform shell of colloidal particles around the core material. Methods described herein provide a stable, robust route to forming colloidosomes.

Moreover, conventional methods to form colloidosomes generally do not result in a stabilized colloidosome structure. Colloidosomes formed from conventional methods lack mechanical strength and require additional steps, such as sintering, chemical coupling and the like, in order to form a colloidosome with acceptable mechanical strength.

In contrast, the attractive interactions reduces or removes the need to stabilize the colloidal particles to each other to maintain the colloidosome structure as was done in conventional methods. The attractive interaction between the colloidal particles and the core material generates a colloidosome structure that has been stabilized during assembly. Accordingly, methods for forming colloidosomes described herein reduce the need to stabilize the colloidosome structure after they have formed at the immiscible fluid interface as is done in conventional colloidosomal systems.

Thirdly, colloidosomes formed from conventional methods do not provide a reliable way of controlling the permeability of the colloidosome. Typically, materials smaller than the interstitial spacing or voids between the colloidal particle layer may pass through, or permeate, into and out of the colloidosome. However, materials larger than the pore sizes do not readily enter into or egress out of the colloidosome. Therefore, in conventional method, the colloidosomes were usually physically broken to permit larger materials to be released from the colloidosome. Such method leads to irreversible destruction of the colloidosome, which can be undesirable in some applications.

Colloidosomes described herein further provide the advantageous characteristic of being able to tune the permeability properties of the colloidosomes. The interstitial space or void space between the colloidal particles can be reversibly controlled, enabling control of the movement of species into and out of the colloidosome structure. Accordingly, the permeability of the colloidosomes described to both small and large molecules can be adjusted and/or selected without irreversibly damaging the colloidosomes structures.

Moreover, in conventional methods, colloidosome size is dictated, in part, by the size of the emulsion droplets. Emulsions are not always monodisperse, which in turn leads to the formation of polydisperse colloidosome populations. Polydisperse colloidisomes may be undesirable in certain applications

In contrast, the core material, such as a hydrogel, can be prepared with a controlled and predictable size so that colloidisomes of uniform size and size distribution can be readily prepared. Monodisperse colloidosomes are important in applications that require a controlled release kinetics of encapsulants and/or adsorbents, such as cosmetic formulations. Methods for preparing gel droplets of precise and uniform size are known and can be used to prepare a monodisperse population of gel core material. For example, capillary-based microfluidics have been used to prepare monodisperse droplet populations.

### Core Material

In certain embodiments, the core material can include a polymer gel. A gel is a form of material between the liquid and solid state. It consists of a crosslinked network of long polymer molecules with liquid molecules trapped within the network. The crosslinks can provide physical strength, although the glass transition temperature of the polymer making up the gel may be below room temperature. The gel can include water (i.e., a hydrogel) and/or organic liquids. Swelling and deswelling in water is a characteristic of a hydrogel.

The gel can be formed as a particle, such as a spherical particle, an ovoid particle, a cylindrical particle, or some other three-dimensionally shaped particle. The gel particle can have at least one dimension that is microns to millimeters in size. The gel particle is generally larger than the colloidal particles. However, the shape and size of the core material is not limited in any particular way. The particle size of the core material is dependent on the intended application of the resultant colloidisomes. Generally, the core material diameter is in the range of about one or more microns to millimeters.

Any conventional method of preparing a particle may be used. Exemplary methods include capillary-based microfluidic techniques, precipitation polymerization techniques, inverse suspension polymerization techniques, and the like. Where uniform particle formation is desired, capillary-based microfluidic techniques similar to the method described in Kim et al., “Fabrication of monodisperse gel shells and functional microgels in microfluidic devices,” Angew. Chem. Int. Ed., Vol. 46, pp. 1819-1822 (2007) and Utada et al., Science, Vol. 308, pp. 537-541 (2005), both of which are incorporated by reference herein in their entireties, may be used.

The gel possesses at least one property that exhibits an attractive interaction with the colloidal particles. For example, the gel can possess electrical charges, magnetic materials, and the like that are capable of attracting the colloidal particles toward the core. The gel may be positively charged through introduction of ionic groups, e.g., cationic or anionic groups. Magnetic material can be included in the gel that attract colloidal particles to the core. For example, the gel can entrain certain magnetic materials (as solids or in solution) within the crosslinked network that are attractive to magnetic metal particles that serve as the colloidal particles.

In one or more embodiments, the core material is responsive to an external stimulus that causes a change in a property in the core material. In certain embodiments, the properties of the core material are altered upon application of an external stimuli, such as a temperature, electric field, magnetic field, pH, ionic strength and the like. Exemplary external stimuli can include physical stimuli, chemical stimuli or combinations of physical and chemical stimuli. Examples of physical stimuli include temperature; electromagnetic radiation, such as infrared energy, visible light and ultraviolet light. Examples of chemical stimuli include concentration of ionic species, pH, crosslinking agents, such as cross-linking agents which crosslink the polymer network of the gel, and solvents.

In one or more embodiments, the core material responds to the external stimulus by undergoing a volumetric change. When a gel undergoes a dramatic change in volume, it is sometimes referred to as a phase change. At the extremes, the gel particle can change from a fully expanded (swollen) gel state to a collapsed (deswollen) solid state. Phase-transition conditions at which the phase-transition gels exhibit a significantly large volume change can include combinations of physical conditions, combinations of chemical conditions, or combinations of physical and chemical conditions.

Temperature-responsive phase transition material (or “thermally responsive” material) undergo a phase transition and/or alter the size of the material in response to thermal energy. For example, the core material may be a polymer, such as a gel, that changes its volume from an expanded state at a lower temperature to a collapsed state at a higher temperatures. Thermosensitive polymer hydrogels contract (deswell) when the temperature is raised above the lower critical solution temperature. The difference in volume between the expanded phase of phase-transition gels and the contracted phase of the phase-transition gels can be orders of magnitude. Examples of phase-transition gels are disclosed in Tanaka et al., U.S. Pat. Nos. 4,732,930, and 5,100,933, the teachings of which are incorporated herein by reference. The polymers of the gel network can comprise natural polymers, synthetic polymers, or cross-linked synthetic and natural polymers. Also, the polymers can be block copolymers. Examples of synthetic polymers include poly(N-isopropylacrylamide), poly(acrylamide), poly(acrylic acid), protein gels, hydroxypropyl cellulose, polyvinylamine, starch, xanthan gum, agar, gelatin, hyaluronic acid, Arabic acids, alginate, and the like.

pH-responsive phase transition material (or “pH responsive” material) undergo a similar phase transition and/or alter the size of the material in response to a change in pH. For example, the core material may be a polymer, such as a gel, that changes the volume of the gel from an expanded state to a collapsed state at higher or lower pH values. In one or more embodiments, the gel may be ionized so that the gel has a net positive or negative charge. Changes in ionic strength of the gel environment, for example by changes in pH or salt concentration, can also bring about a change in the volume of the gel. pH sensitive hydrogels may include polypeptide hydrogels made of hydrophobic (e.g., leucine) and hydrophilic (e.g., gluatamine) amino acids, poly(N-isopropylacrylamide), poly(acrylamide), and poly(acrylic acid). Similar effects are observed in the change of the ionic strength of the solution, for example, by the addition of salts, such as sodium chloride, calcium chloride, magnesium chloride, sodium carbonate, cupric chloride, and the like.

Some exemplary electrically-responsive material can include materials containing electrically responsive elements that can deform or alter the size of the material in response to an applied electric field. For example, the core material may include a polymer, such as a gel, having electrically responsive elements that changes the volume of the gel from a collapsed state to an expanded state upon application, removal, increase, or decrease of an electric field. Exemplary materials that can be utilized include N-isopropyl acrylamide, vinyl alcohol, vinyl amine, acrylic acid, gelatin, urethane, vinylsulfonic acid, and the like.

### Colloidal Particles

Any suitable colloidal particles can be utilized. In certain embodiments, the colloidal particles can include a metal, a semiconductor, a polymer, an inorganic material, and the like. In some other embodiments, the colloidal particles can include nanometer sized particles (also referred to herein as “nanoparticles”), micrometer sized particles (also referred to herein as “microparticles”), and/or the like. The colloidal particles have a particle size that is smaller than that of the core material.

The colloidal particles possess an attractive interaction with the core material. For example, the colloidal particles possess electrical charges, magnetic properties, and the like that are attractive to the core material. In one or more embodiments, the colloidal particles may be negatively charged through introduction of ionic groups when the core material is positively charged. Alternatively, attractive interactions may arise through electrostatic interactions. Some exemplary semiconductor colloidal particles include silicon, germanium, gallium arsenide, cadmium selenide, and the like particles. Some exemplary polymer colloidal particles include polystyrene, polymethyl methacrylate, poly(ε-caprolactone), poly(lactic acid), poly(lactic acid-co-glycolic acid), and the like particles. Some exemplary inorganic colloidal particles include gold, silver, copper, cobalt, palladium, platinum, manganese-zinc, nickel-zinc, iron-platinum, silica, titania, iron oxide, zinc oxide, nickel oxide, and the like particles.

In one or more embodiments, the colloidal particles can possess magnetic properties that can be attracted to a gel having a magnetic material. For example, the gel can contain certain magnetic particles within the crosslinked network and the colloidal particles can include a magnetic material. Exemplary magnetic colloidal particles include gold, silver, copper, iron oxide, manganize-zinc, nickel-zinc, nickel oxide, cobalt, iron-platinum, CoFe₂O₄, and the like particles.

In certain embodiments, the colloidal particles can possess additional properties, such as fluorescence and the like, that are suitable for certain chemical, biological, and the like applications. For example, the colloidal particles might be sensitive to certain diagnostic tools (e.g, MRI, ultrasound, x-ray, etc.) to determine the location of the colloidosomes, e.g., colloidisomes that encapsulate a drug or other therapeutic agent, in a human body.

### Methods to Form Colloidosomes

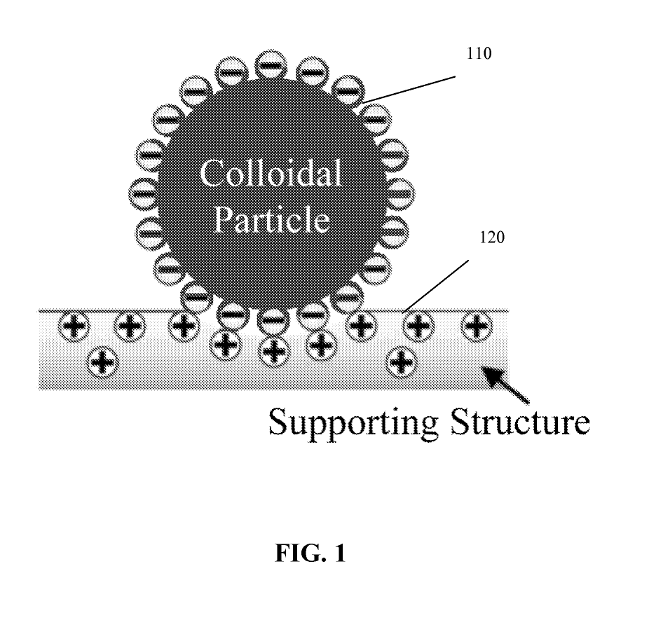

In certain embodiments, colloidosomes can be formed by assembling colloidal particles on a surface of a core material, such as a gel or hydrogel particle, to form one or more shells of colloidal particles. Any particles having at least one attractive interactions with the core material can be utilized. In certain embodiments, the attractive interactions may be stronger than thermal fluctuations that exist during or after the formation of colloidosomes so that the particles are and remain bound to the core material. For example, as schematically illustrated in FIG. 1, the core material 120 can be a positively charged gel while the colloidal particles 110 can be negatively charged. As another example, the gel can include magnetic materials that attract metallic colloidal particles. As will be readily apparent to one of ordinary skill in the art, any one or combination of attractive interactions between the core material and the colloidal particles can be utilized to form colloidosomes.

In certain embodiments, the colloidal particles and the core material can be combined in any suitable manner that results in the formation of a shell of colloidal particles around the core material. For example, the colloidal particles and the core material having attractive interactions with each other can be placed in a liquid medium, such as water, oil, organic solvents, and the like, to allow the colloidal particles to form a shell around the core material. The colloidosome remains stable without the having to lock, sinter, or fuse the particles onto each other, although the colloidal particles can be locked, sintered, or fused together if so desired.

In some embodiments, the colloidal particles and the core material can be placed in an environment that achieves or further enhances the attractive interactions. For example, the colloidal particles and the core material can be placed in a buffer solution that enhances the negative and positive charges on the respective materials. For example, if the gel contains carboxylic groups, the gel can be placed in a basic buffer solution (e.g., pH greater than 7), which can increase the amount of negative charges. Other variables that can be adjusted include, but are not limited to, temperature, pressure, applied electric field, applied magnetic field, and the like. For example, pressure can be applied during the formation of colloidosomes to “push” the particles onto the gel, effectively increasing the attractive interactions. Temperature can be increased so that the molecular mobility of the gel increases, leading to an apparent reduction in the attractive interactions. Similarly, electric and magnetic fields can be applied to change electric and/or magnetic force the gel and colloidal particles experiences, thereby effectively altering the attractive interactions experienced by the gel and colloidal particles.

### Tuning the Properties of the Colloidosome

Once the colloidal particles are assembled around the core material, the colloidosome can be exposed to one or more external stimulus to alter the properties of the colloidisomes. The colloidosome can include a component that is responsive to an external stimuli and which can invoke a change in a property of the colloidosome, once stimulated. For example, permeability, mechanical properties and morphology of the colloidal layer can be altered.

In one aspect of the invention, the packing uniformity, packing density and particle layering on the core material can be altered by changing the size of the core materials after assembly of the colloid particles around it. In this manner, permeability of a colloidosome can be tuned by changing the morphology of the colloidosome. As used herein, “permeability” refers to permeation or movement of one or more species into and out of the colloidosome.

Tuning the colloidosome may occur during the manufacture of the colloidosome, for example, to increase the packing density of the colloidal particle shell and thereby reduce the permeability of the colloidosome. This may be desirable, for example, to reduce premature permeation and loss of a substance that has been encapsulated within the colloidosome. Tuning the colloidosome may occur during use, for example, to increase volume size of the gel core, thereby reducing the packing density of the colloidal particle shell and thereby increasing the permeability of the colloidosome.

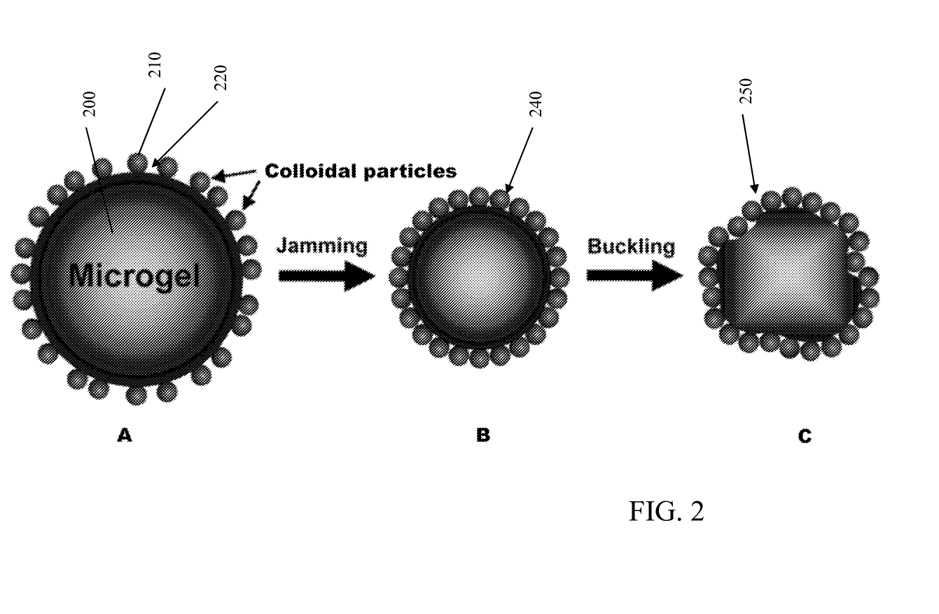

FIG. 2A shows a colloidosome having a thermally responsive gel 200 as the core material and colloidal particles 210 that are loosely spaced on the surface of the gel core. Upon assembly, the colloidal particles are attracted to the gel core, but may be assembled in a random manner where the interparticle distances 220 between the colloidal particles are relatively high, as shown in FIG. 2A. In the state shown in FIG. 2A, the permeability into and out of the gel is expected to be very high due to the large spacing between the colloidal particles. Due to the large spacing, both large and small molecules may migrate into and out of the colloidosome.

Upon application of heat, the gel can shrink leading to a reduced spacing 240 between colloidal particles as shown in FIG. 2B. In one embodiment, the gel can shrink to about 90%, or about 80%, or about 60% or up to 20% of its original size. The colloidosome shown in FIG. 2B is expected to exhibit lower permeability than that shown in FIG. 2A. The closer spacing of the colloidal particles reduces the interstitial distances in the colloidal layer and lowers the permeability of the colloidal particle layer. It may be expected that higher molecular weight particles do not move as readily across the colloidosome boundary layer. In certain embodiments, the colloidosome shown in FIG. 2B can even inhibit permeability of certain species that are larger than the interstitial voids between the colloidal particles.

Even further reduction in permeability may be achieved by further heating of the gel or by application of yet another external stimulus that serves to further reduce the core material volume. In some embodiments, In one embodiment, the gel can shrink to about 80%, or about 70%, or about 50% or up to 10% of its original size. Upon further application of heat, buckling of the colloidosome structure can occur leading to a structure 250 shown in FIG. 2C. As shown, further crowding of the colloidal particles around the reduced surface area of the core material results in reduced spacing between particles and/or overlap of particles on the core surface. Accordingly, a more tortuous pathway for the species permeating through the colloidal particles can be obtained, and the colloidosome shown in FIG. 2C is expected to exhibit lower permeability than that shown in FIG. 2B.

Lowering the temperature can return the structure of the colloidosome to that shown in FIG. 2A or 2B, which can again increase the interparticle distance between the colloidal particles. Accordingly, the colloidosomes described herein allow a precise control of the permeability by applying certain external stimulus. In particular, the effects can be reversible and the colloidosome can cycle reversibly between both expanded, collapsed and buckled states.

The tunable property of the colloidosome described herein can be utilized to capture desired molecules, such as drugs, inside the core material until they are desirably released. For example, the core of the colloidosome can be infused with desired molecules in a state shown in FIG. 2A. In other embodiments, the desired molecules can be introduced into the gel core of the colloidosome before assembly of the colloidosome. After the desired molecule has permeated into the core, an external stimulus can be applied to change the morphology of the colloidosome to a state shown in FIG. 2B or 2C, which can allow the molecules to be trapped inside the colloidosome core. Thereafter, the colloidosome having the trapped molecules can be delivered to a desired location and the molecules can be released by returning the colloidosome structure to that shown in FIG. 2A by an application of an external stimulus.

Other methods for tuning the properties of the colloidosomes, are within the scope of this application. For example, the permeability of the colloidosomes can be tailored by changing the size and/or amount of the colloidal particles. The thickness of the colloidal shell can also be tailored to control the permeability. For example, the thickness of the colloidal shell can be increased by an alternating deposition of negatively-charged and positively-charged colloidal particles. The permeability can further be controlled by altering the degree of fusion between the colloidal particles. For example, when utilizing colloidal particles composed of a glassy polymer, heat can be applied to allow the particles to flow and fuse together. Many other methods for tuning the properties of the colloidosomes will be readily apparent to one of ordinary skill in the art.

### Applications

The colloidosomes described herein have wide ranging applications in the field of drug delivery, cosmetic delivery, food delivery, LCD display devices, polymer blends, paints, and the like.

In one or more embodiments, the gel core may further include additional components selected to achieve an intended purpose or application of the gel. By way of example, the gel may include a drug or therapeutic agent. The drug-encapsulating colloidosome can be used in drug delivery applications.

For example, the colloidal particles can be biocompatible particles, such as poly(lactic acid-co-glycolic acid) nanoparticles having certain electrical charges. Moreover, the colloidal particles can be treated with a variety of functional biopolymers, such as proteins, enzymes, and the like, to impart a “smart” behavior wherein the functional biopolymers allow the activation of release of encapsulated molecules in the vicinity of corresponding/complementary proteins, enzymes, and the like. The functional biopolymers can further act as a targeting agent, wherein the functional biopolymer acts to direct the colloidosomes to a desired location. Accordingly, the resulting colloidosomes can provide an integrated mechanism for targeted delivery of the colloidosomes, controlled release of the encapsulated material, and biocompatibility with the subject.

In one or more embodiments, a colloidosome is prepared in the expanded gel state. A drug is allowed to permeate into the gel core from the surrounding solution. Alternatively, the drug can be introduced into the gel before particle formation, thereby entraining the drug in the particle core. Once entrained, the colloidosome can be heated, causing the colloidosome to collapse and encapsulate the drug in the colloidosome interior. Upon cooling, the particle expands, permitting the drug to be released from the colloidosome.

A similar effect may be achieved by altering the pH. For example, a gel core may be selected that is in a collapsed state has low pH (e.g., stomach pH) and which expands to a swollen state at lower pH (e.g., gut pH) to preferentially release the drug in the intestines.

In one or more embodiments, the colloidal particles may be selected to include properties that promote the intended application of the colloidosome. By way of the example, the colloidal particles may be selected for their resistance to permeation of a certain material. For example, the colloidal particles can be positively charged silica colloidal particles that are resistant to organic (non-polar or low polar) pharmaceutical drugs that permeate into and out of the gel core material.

### EXAMPLE

### Fabrication

Monodisperse temperature-responsive gel particles were fabricated as follows: Poly(N-isopropylacrylamide)-based gel particles were synthesized using a capillary-based microfluidic method, similar to the method described in Kim et al., “Fabrication of monodisperse gel shells and functional microgels in microfluidic devices,” Angew. Chem. Int. Ed., Vol. 46, pp. 1819-1822 (2007) and Utada et al., Science, Vol. 308, pp. 537-541 (2005), both of which are incorporated by reference herein in their entireties.

A capillary microfluidic device was fabricated by assembling two round capillaries (1 mm outer diameter, 0.58 mm inner diameter, borosilicate, World Precision) and a square capillary (1 mm inner diameter). Three types of fluids, outer, middle, and inner fluids, were flowed into the capillaries. Dimethyl phenylmethylsiloxane (DC #550, viscosity=125 mPa·s, density=1.06 g/mL) was utilized as the outer fluid. Aqueous monomer solution containing N-isopropylacrylamide (15.5% w/v), N,N′-methylenebisacrylamide (0.55% w/v), N,N,N′,N′-tetramethylethylenediamine (2 vol %), and [2-(methacryloyloxy)ethyl trimethyl ammonium chloride (1.5 vol %) was used as the middle fluid. Aqueous ammonium persulfate (3% w/v) solution was used as the inner fluid. Controlling the flow rates of each fluid generated uniform pre-gel droplets, which were then polymerized in situ with a redox reaction at room temperature to form gel particles. Using this technique, gel particles having a diameters of about 50, 120, and 620 μm were synthesized. The synthesized gel particles were positively-charged.

Negatively-charged polystyrene (PS) particles (sulphate-functionalized and carboxylate-functionalized) having diameters ranging from about 20 nm to about 4 μm were purchased from INVITROGEN.

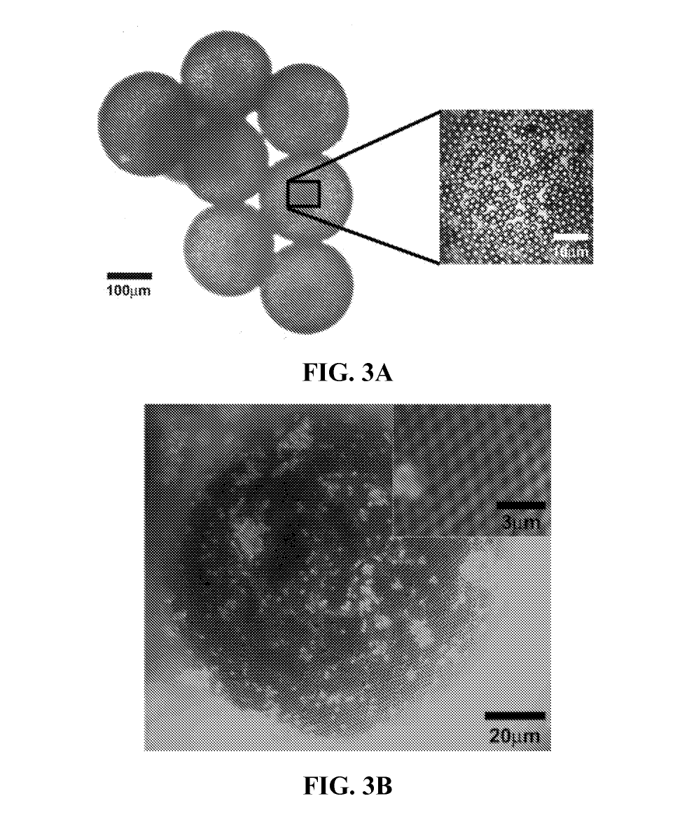

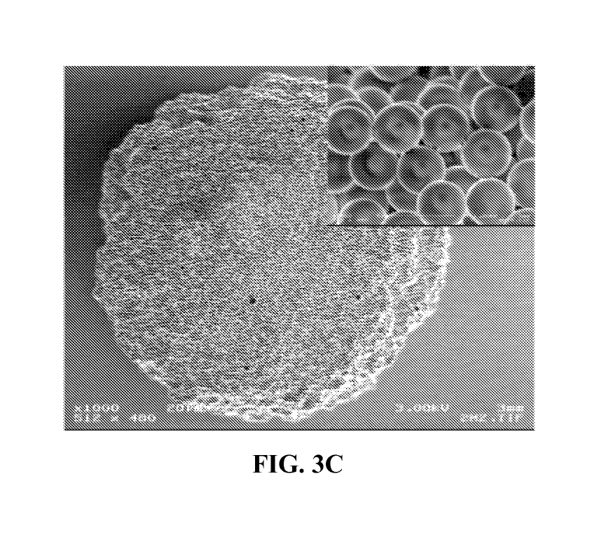

Self-assembled colloidisomes were prepared using various combinations of gel particles and PS particles. Desired gel particles and PS particle sizes were selected. Aqueous dispersion of the positively charged gel particles were cooled to 5° C. Excess amounts of the negatively-charged PS particles were added to the cooled gel dispersion and vigorously mixed for about 1 minute, where the electrostatic interactions cause the PS particles to adsorb onto the gel to form colloidosomes. The resulting colloidosomes were collected by repeated centrifugation with large amounts of deionized water, which removes the excess PS particles in the liquid phase. An exemplary image of a colloidosome prepared according to this method is shown in FIG. 3A. As shown in FIG. 3A, initially, the PS particles only partially covered the gel surface clearly visible in the inset in FIG. 3A. Without wishing to be bound by theory, the partial coverage may be due to the electrostatic repulsion that could occur between the PS particles.

### Morphology

The morphology and phase behavior of the colloidosomes were altered by changing the temperature of the colloidosomes. The colloidosomes were placed in a glass container on a temperature-controllable hot stage (PHYSITEMP, TS-4 ER) and the temperature of the colloidosomes were raised from room temperature to 65° C.

Increasing the temperature caused the volume of the gel particle to decrease from about 200 μm to about 150 μm, leading to a reduction in the average spacing between the adsorbed colloidal PS particles. As shown in FIG. 3B, the adsorbed PS particles eventually formed a single packed layer with some crystalline order (clearly visible in the inset to FIG. 3B). The thickness of the single packed layer varied linearly with the diameter of the utilized PS particles.

As shown in FIG. 3C, further increase of the temperature further decreased the size of the gel particle core from about 150 μm to about 100 μm and causing the colloidal layer to buckle and distort. The PS particles formed a multilayered buckled shell, creating a dense coating of PS particles (clearly visible in the inset to FIG. 3C). Moreover, the electron micrographs show that some gel may be present between the PS particles. Total decrease in diameter for these colloidosomes was about 40%.

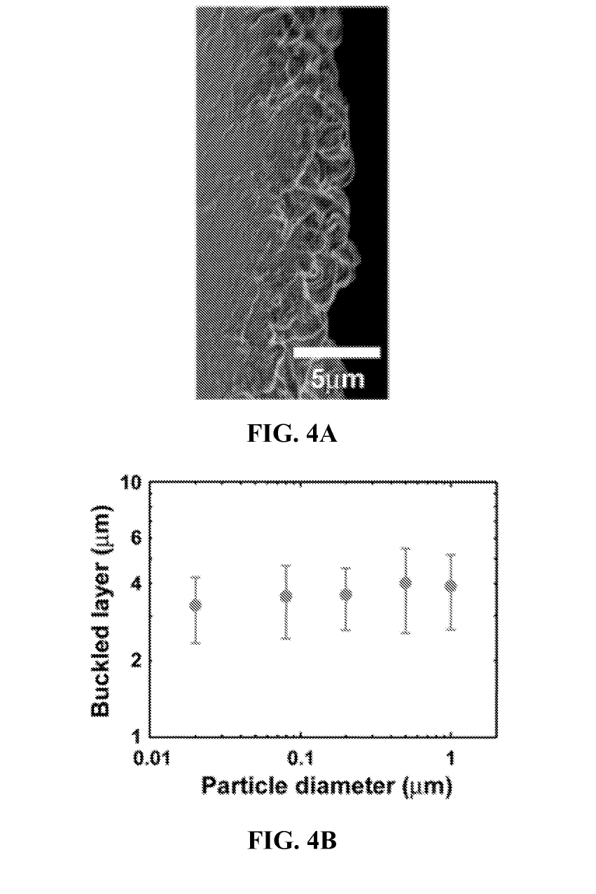

To measure the thickness of the buckled shell, the buckled colloidosomes assembled using PS particles of particles sizes ranging from 20 nm to 1 micron were dried and fractured. FIG. 4A shows a scanning photomicrograph of a cross-section of an exemplary buckled colloidosome. As shown in FIG. 4B, the thickness of the buckled shell was insensitive to the size of the adsorbed PS particles. Without wishing to be bound by any particular theory or mode of operation, this may indicate that the buckling process is determined predominantly by the decrease in surface area of the gel particles as they shrink irrespective of the colloidal particle size.

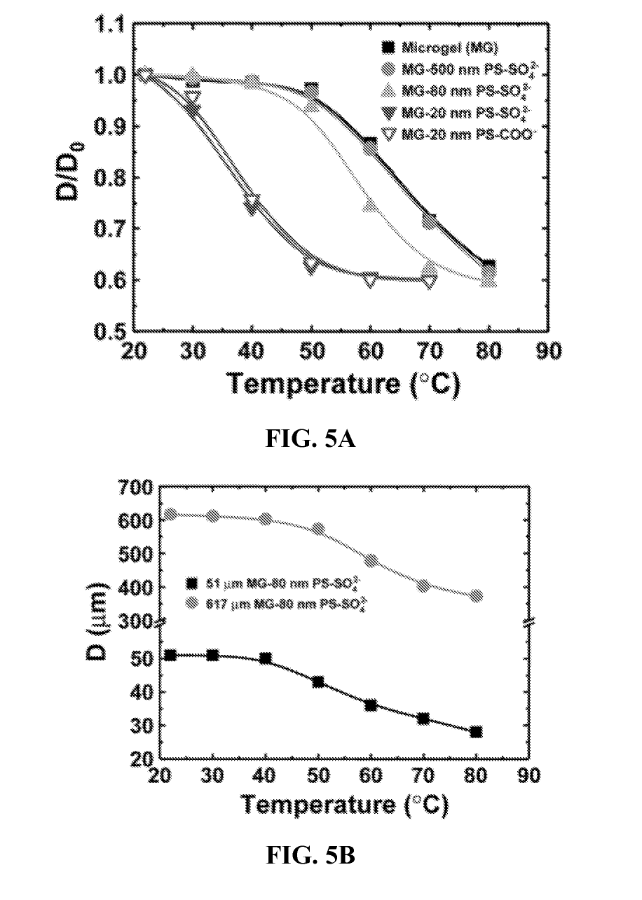

The temperature at which buckling of the colloidosomes occurs varied with the colloidal particle size. Gel cores without any colloidal PS particles and colloidosomes having 500 nm, 80 nm, and 20 nm PS particles were studied. The PS colloidal particles were negatively charged with sulfate groups. Colloidosomes having 20 nm PS particles negatively charged with carboxylate groups were also studied. As shown in FIG. 5A, 500 nm PS nanoparticles did not affect the buckling temperature (the colloidosomes had the same buckling profile as the uncoated gel particle), while 80 nm PS nanoparticles reduced the buckling temperature by about 10° C., and 20 nm PS nanoparticles reduced the buckling temperature by about 30° C. This effect was independent of the type of ionic groups responsible for the charge on the PS particles, as identical behavior was observed with 20 nm sulphate-functionalized PS particles and 20 nm carboxylate-functionalized PS particles. Moreover, as shown in FIG. 5B, the buckling temperature for colloidosomes made from different size of gel particles, but made from the same sized PS particles, did not change.

### Permeability

The colloidosomes were immersed in a flowing aqueous solution containing fluorescein molecules and the fluorescence intensities inside the colloidosomes were measured as a function of time. It should be noted that the fluorescein is negatively charged and the gel particle is positively charged. Accordingly, there may be some attractive interaction between the fluorescein and the gel particle.

The results show that fluorescein molecules readily pass through the colloid shells in the initial state. In contrast, when the colloidosomes have buckled, the permeability decreases significantly. The reduction in permeability is also noticeably much higher in colloidosomes using smaller-sized colloidal particles.

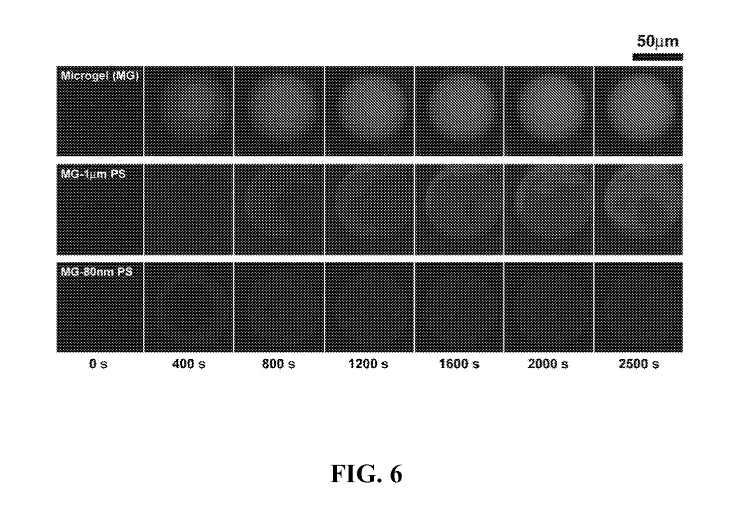

Dilute aqueous fluorescein sodium salt solution 90.5 μM) in a square glass capillary tube (1 mm inner dimension) was set at 500 μL/h, using a syringe pump. Experiments were conducted at 65° C., set by a temperature-controlled stage on a fluorescence microscope. As shown in FIG. 6, fluorescein readily diffused into the gel particle without any colloids (see top row) while no noticeable diffusion occurred for buckled colloidosomes having a 80 nm PS nanoparticle layer (see bottom row). The buckled colloidosomes having 1 μm PS particles (see middle row) exhibit permeability that is in between the uncoated gel particle and the colloidosome having an 80 nm PS nanoparticle layer. Without wishing to be bound by theory, the buckled colloidosomes may have a dense, complex colloidal layers that block transport of the fluorescein molecules into the gel particle.

Cooling the buckled colloidosomes increased the permeability of the fluorescein molecules into the gel particle. Without wishing to be bound by theory, cooling may lead to expansion of the gel particle, leading to increased spacing between the colloidal particles. In return, this may allow the negatively-charged fluorescein molecules to be more easily permeate into the positively-charged gel particle.

### Permeability Control Using Soft Colloidal Particles

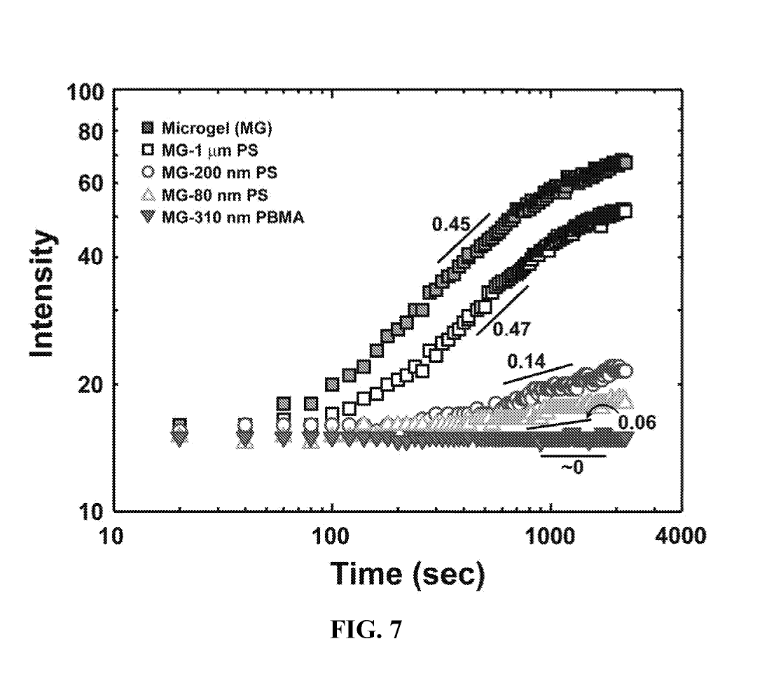

As described above, an alternative method of controlling the permeability of colloidosomes can include controlling the degree of fusion between the colloidal particles. In certain cases, fusion of the colloidal particles can be induced by applying stress at temperatures that is near or above the glass transition temperature of the colloid. Accordingly, negatively-charged poly(butyl methacrylate) (PBMA) particles, having a glass transition temperature of about 30° C., was utilized instead of the PS colloidal particles. The average diameter of the PBMA particles was about 310 nm. The PBMA particles appear to have packed more closely on the gel surface, possibly due to their deformability. As a result, the diffusion of fluorescein into the gel was inhibited, even when compared to colloidosomes having 80 nm PS particles (see FIG. 7). In fact, permeability of fluorescein molecules appears to be inhibited even without any buckling of the colloidosomes.

Upon review of the description and embodiments of the present invention, those skilled in the art will understand that modifications and equivalent substitutions may be performed in carrying out the invention without departing from the essence of the invention. Thus, the invention is not meant to be limiting by the embodiments described explicitly above, and is limited only by the claims which follow.

What is claimed is:

1. A colloidosome comprising:
a core polymer gel material and a collection of colloidal particles assembled on a surface of the core material, wherein the core polymer gel material surface and the colloidal particles possess at least one attractive interaction, wherein the core polymer gel material is responsive to an external stimulus that alters the interparticle distance between colloidal particles.

2. The colloidosome of claim 1, wherein the core material has an initial diameter and the interparticle distance is altered by changing the initial diameter of the core material to a new diameter.

3. The colloidosome of claim 1, wherein the core material is responsive to at least one of heat, electric field, pH, and magnetic field.

4. The colloidosome of claim 1, wherein the attractive interaction is an attractive electrostatic interaction, attractive ionic interaction, attractive magnetic interaction, attractive pyroelectric interaction, or combinations thereof.

5. The colloidosome of claim 1, wherein the core material is a thermally responsive hydrogel.

6. The colloidosome of claim 1, wherein the core material is a pH responsive hydrogel.

7. The colloidosome of claim 1, wherein the core material is a positively charged hydrogel particle.

8. The colloidosome of claim 7, wherein the colloidal particles are negatively charged.

9. The colloidosome of claim 1, wherein the particles are selected from the group consisting of a metal, a semiconductor, a polymer, an inorganic material, and combinations thereof.

10. The colloidosome of claim 1, wherein the colloidal particles have diameters that range from about a nanometer to about a millimeter.

11. The colloidosome of claim 10, wherein the size of the core material is larger than the size of the colloidal particles.

12. A method for making a colloidosome, the method comprising:
exposing a core gel particle to a plurality of colloidal particles to form a shell of colloidal particles on a core material;
wherein the core gel particle and the colloidal particles have at least one attractive interaction; and
wherein the core gel particle is responsive to an external stimulus to change the interparticle distance between the colloidal particles.

13. The method of claim 11, further comprising:
forming a hydrogel particle as the core material.

14. The method of claim 11, further comprising:
exposing the colloidal particles and the core gel particle to an environment that enhances the attractive interaction between the colloidal particles and the core gel particle.

15. The method of claim 11, wherein the forming a shell of colloidal particles comprises placing the core material and the colloidal particles in a liquid medium and mixing the liquid medium containing the core material and the colloidal particles.

16. The method of claim 14, wherein the liquid medium comprises water.

17. A method for changing at least one property of a colloidosome, the method comprising:
applying one or more external stimuli to a colloidosome, said colloidosome comprising a core gel material and a shell of colloidal particles formed on the core gel material, wherein the core material and the colloidal particles possess at least one attractive interaction, and wherein the core material responds to the one or more external stimuli to change the interparticle distance between the colloidal particles.

18. The method of claim 16, wherein the change in interparticle distance of the colloidosome particles causes a change in permeability of the colloidosome.

19. The method of claim 18, wherein the morphology of the colloidosome is changed by expansion or contraction of the core gel material.

20. The method of claim 19, wherein the core material comprises a hydrogel, the colloidal particles comprise polymeric particles, and the external stimulus comprises heat.

21. A drug delivery vehicle, comprising:
a core polymer gel material and a collection of colloidal particles assembled on a surface of the core material, wherein the core polymer gel material surface and the colloidal particles possess at least one attractive interaction, wherein the core polymer gel material is responsive to an external stimulus that alters the interparticle distance between colloidal particles; and
an active agent encapsulated in the core polymer gel material.

22. The drug delivery vehicle of claim 21, wherein the colloidal particles have a polarity that tends to repel the active agent.

23. The drug delivery vehicle of claim 21, wherein the collection of colloidal particles comprise biofunctional materials.

24. The drug delivery vehicle of claim 23, wherein the collection of colloidal particles comprise proteins and enzymes attached to at least a portion of the surface of the colloidal particles.
